(19) United States
(12) Patent Application Publication
(10) Pub. No.: US 2021/0208081 A1
(43) Pub. Date: Jul. 8, 2021

(54) ANALYSIS OF URINE TEST STRIPS WITH MOBILE CAMERA ANALYSYS AND PROVIDING RECOMMENDATION BY CUSTOMISING DATA

(71) Applicant: VIVOSENS BIYOTEKNOLOJI AR-GE SANAYI & TICARET LTD. STI., Istanbul (TR)

(72) Inventors: Miray TAYFUN, Istanbul (TR); George RADMAN, Melbourne (AU)

(73) Assignee: Vivosens Inc, San Francisco, CA (US)

(21) Appl. No.: 17/052,533

(22) PCT Filed: May 15, 2018
(86) PCT No.: PCT/TR2018/050233

§ 371 Date: Nov. 3, 2020

(51) Int. Cl. G01N 21/78; G01N 33/493; G06T 7/00; G06T 7/40

(52) U.S. Cl. CPC G01N 21/78; G01N 33/493; G01N 2021/7759; G06T 7/40; G06T 7/0014

(57) ABSTRACT

The present invention relates to a method of analyzing a urinalysis wherein an image processing system is used to recognize a urine test strip (1) and the area of said urine test strip (1) and the segments therein, and also colors of every segment and their color changes with the cameras of intelligent devices, and which is configured such as to convert the data received from the image processing system to significant data.

### TECHNICAL FIELD

The present invention relates to a method of urine analysis wherein an image processing system is used to recognize a urine test strip and the area of said urine test strip and the segments therein, and also colors of every segment and their color changes with the cameras of intelligent devices, and which is configured such as to convert the data received from the image processing system to significant data.

### PRIOR ART

Numerous analysis methods are used in detecting diseases in the field of medicine. Urine analysis is one of the analysis methods that is conducted most commonly and routinely as well as for almost every patient.

A method commonly used in urinalysis processes includes analyzing the urine spilled over a urine test strip or the color changes occurring by urine on the strip by dipping the urine test strips into a semi-closed container containing the urine. In the recognition systems in which the skilled persons in the art carry out the process without using any auxiliary equipment, the analysis method can be accomplished depending on the ability of observation of a skilled person in the art. In other words, a skilled person is always required for conducting urinalysis processes smoothly, thus it is not possible to be performed under any condition. Furthermore, as it is an empirical process, it is very susceptible to any possible fault.

There are certain nonempirical analysis methods as well, however a method that enables a urinalysis to be carried out without any extra costs in every environment has not been provided yet.

In a prior art United States patent document numbered U.S. Pat. No. 8,655,009B2, it is disclosed that the color-based reaction tests of the related biological materials are performed, in an uncalibrated environment, by capturing a digital image of a test strip together with a reference color chart adjacent thereto or a color chart on a strip. The biological materials used may include a urine, blood specimen and the like. The image data that represent the test pads in the test strips and the reference color blocks in the reference chart would be present on the picture to be subsequently taken, and would be compared to determine the color matches between the test pads and the related pixels. Based on this comparison process, a range of test results can be obtained, which are effectively capable of recognizing which color blocks from the reference chart is the most compatible with the test pads of the related test strip. The test results obtained can be subsequently delivered to users in a printed or visual manner. Alternatively, the test results can be kept to be taken later.

In another prior art United States patent document numbered US2015254844A1, a method for a calculating device with an imaging device to read the test strip of a specimen is disclosed. The method includes capturing an image of the test strip of the specimen. Wherein the image includes a reaction area, a color calibration area, and a temperature calibration area on the test strip of the specimen. A color of the reaction area is determined based on one or more than one color of the same. The color of the reaction area is associated with a color value of the color calibration area or the reaction area.

A further prior art Korean patent document numbered KR101124273 describes a urinalysis system that works with the image processing technology that enables a color change in the segments of a urine test strip and recognizing the color changed, and this method developed processes the color changes by converting them to a significant data. A server processes a urine recognition data, and thus it would be possible for a patient to use the urinalysis system conveniently.

When the prior art methods are taken into consideration, a need for developing an image processing system to be used to recognize a urine test strip and the area of said urine test strip and the segments therein, and also colors of every segment and their color changes with the cameras of intelligent devices as well as a urinalysis method configured such as to convert the data received from the image processing system to a significant data has risen.

### AIMS AND THE SUMMARY OF THE INVENTION

An aim of the invention is to provide a method of analyzing urine test strips by analyzing the same with a mobile camera.

Another aim of the invention is to provide a method wherein a strip with at least a reaction area and at least a frame area enclosing said reaction area is used.

A further aim of the invention is to provide an analysis method that is capable of converting the analysis results obtained, following the analysis of urine test strips by analyzing with a mobile camera and converting said analysis results to a recommendation.

The invention relates to an analysis method of a urinalysis, comprising the following steps of,
pouring a urine sample of a user subjected to a urinalysis over at least reaction areas of a urine test strip such that it is in contact with said areas,
taking an image of the urine test strip enabled to contact with the urine by means of a display unit,
transmitting the image of the urine test strip taken, to a processing unit,
determining at least a reaction area of the urine test strip present on the image taken by using the data in a reference chart provided in a memory unit by the processing unit,
determining the color changes that has occurred in the reaction area of the urine test strip that is selected,
obtaining a urinalysis result by matching the data on the color changes determined with the data contained within the color scale of the memory unit in the processing unit,
receiving the data of the user subjected to the analysis in the user profile from the user data contained within the memory unit by the processing unit,
determining the recommendation in which the user data received and said urinalysis results are matched in a recommendation pool,
submitting the recommendation determined to the user.

Said urine test strip comprises at least a reaction area and at least a frame area enclosing the said reaction area.

Said mobile communication device comprises a memory unit containing the predetermined user data and the data on the reference chart, the color scale and the recommendation pool.

Said mobile communication device comprises an image capturing unit that allows an image of the strip to be taken.

Said mobile communication device comprises a processing unit that generates a urinalysis result by comparing the colors of the reaction area on the image of the urine test strip received from the image capturing unit with the data on the color scale received from the memory unit as well as detecting the matches.

The mobile communication device generates a recommendation, depending on the user data from the said recommendation pool, by matching the urinalysis results of the processing unit with the user data that it takes from the memory unit.

Thus, it would be enabled for the user to conveniently have a urinalysis in any environment with the help of mobile communication, and to receive a recommendation following the examination of their results.

Said recommendation pool is configured such as to be able to provide a recommendation depending on the urinalysis result and the user data. Thus, it is enabled to deliver users recommendation(s) that at least partially contributes to the health of the user based on the analysis results of the user and the data in the user profile.

Furthermore, it would be possible to minimize the mistakes on the image taken from the image capturing unit by preventing the background from flashing when processing the image by ensuring that the frame areas are the same color.

### DETAILED DESCRIPTION OF THE INVENTION

The components that are used in the analysis method of urinalysis provided in order to achieve the aims of the present invention are presented in the accompanying drawings.

In the drawings;

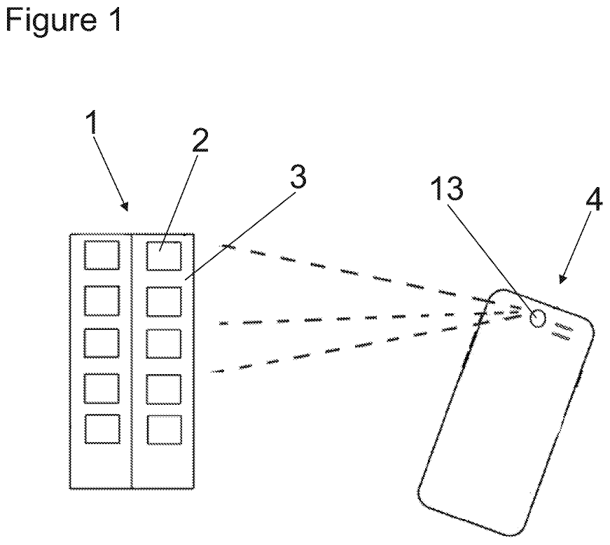

FIG. 1 is a representative view of a urine test strip and a mobile communication device used in the analysis method of urinalysis according to the invention.

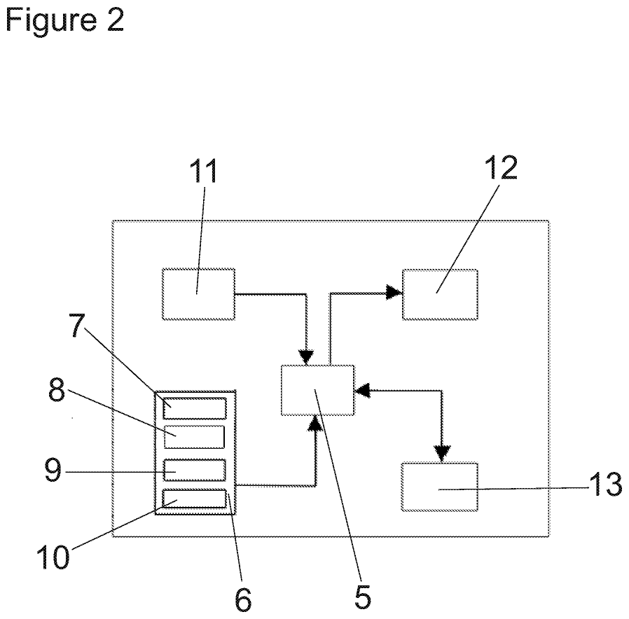

FIG. 2 is a representative view of the units containing the mobile communication device used in the analysis method of urinalysis according to the invention.

The components in the figures are separately numbered and the corresponding definitions of these numbers are as follows.
1. Urine test strip
2. Reaction area
3. Frame area
4. Mobile communication device
5. Processing unit
6. Memory unit
7. Reference chart
8. Color scale
9. Recommendation pool
10. User data
11. Communication unit
12. Display
13. Image capturing unit

The invention relates to an analysis method of a urinalysis, comprising the following steps of,
pouring a urine sample of a user subjected to a urinalysis over at least one reaction area (2) of a urine test strip (1) such that it is in contact with said area,
taking an image of the urine test strip (1) enabled to contact with the urine with an image capturing unit (13),
transmitting the image of the urine test strip (1) taken to a processing unit (5),
recognizing the urine test strip (1) whose image is rendered by using the data in a reference chart (7) provided in a memory unit (6) by the processing unit (5) in the pattern of two columns and five rows, determining 10 reaction areas (2) on the strip,
determining the color changes occurred in the reaction areas (2) of the urine test strip (1) selected,
obtaining a urinalysis result by matching the data on the color changes determined with the data contained within a color scale (8) of the memory unit (6) in the processing unit (5),
receiving the data of the user subjected to the analysis in the user profile from user data (10) contained within the memory unit (6) by means of the processing unit (5),
determining the recommendation in which the user data received and said urinalysis results are matched in a recommendation pool (9),
submitting the recommendation determined to the user.

A urine sample of a user subjected to a urinalysis is poured over at least a reaction area (2) of a urine test strip (1) such that it can penetrate into said strip. Then, an image of the urine test strip (1) is taken with an image capturing unit (13) of a mobile communication device (4). The image taken is transmitted to a processing unit (5) by the image capturing unit (13). The processing unit (5) determines the urine test strip (1) by receiving the data in a reference chart (7) part provided in a memory unit (6) together with the image transmitted.

Locations of the reaction areas (2) on the image of the urine test strip (1) determined are detected by matching the data provided in the reference chart (7) with their mathematical coordinates.

The color change in the reaction areas (2) of the urine test strip (1) matched with the reference chart (7) is analyzed by using the method of RGB histogram in the processing unit (5). Then, the color occurred/unchanged in each reaction area (2) is compared with a different color space again and the colors are recognized by eliminating the characteristics such as flashing, light intensity based on the differences defined relative to distance. The color changes occurred in each reaction area (2) are listed. A urinalysis result is obtained by analyzing the responses occurred by the color change in the reaction area (2) with the predetermined data.

Then, a recommendation is submitted to the user by using the data provided in a recommendation pool (9), and comparing the urinalysis results with the user data (10) kept in the memory unit (6) containing the data related to the user subjected to the urinalysis.

The image capturing unit (13) used in the said method is a camera.

The urine test strip (1) used in said method comprises at least one reaction area (2). Said reaction areas (2) are enclosed by at least one frame area (3). The reaction areas (2) are configured such as to change color by reacting with the urine that contacts to their surfaces. In an embodiment of the invention, the urine test strip (1) comprises a measuring area and a retaining portion having reaction areas (2) in two columns and five rows.

The frame area (3) in the invention is black in color. That the frame area (3) is black in color prevents flashing during the image rendering process. Furthermore, as the colors provided on the dark color are easy to perceive, the sensitivity of image rendering is promoted. Additionally, the reaction areas (2) have the frame areas (3) there between and the portion on which the reaction occurs is separated in parts. Thus, the reaction areas (2) are prevented from being disintegrated with the urine pressure and the image rendering is thereby facilitated.

A new design is developed with the analysis method of urinalysis according to the invention such that the faults of the urine test strip (1) in image rendering is minimized. In addition to this, a urinalysis can be carried out without any need for the high-quality image capturing units (13) thanks to the method developed.

The mobile communication device (4) used in said method is configured such as to communicate with other devices via a communication unit (11). The mobile communication device (4) has a display (12) that allows image display. The data provided from the display (12) is transmitted to a processing unit (5).

Said processing unit (5) and a memory unit (6) contained in the mobile communication device (4) are associated such that they can make data exchange with each other. Said memory unit (6) contains at least the reference chart (7), the color scale (8), user data (10) and data from the recommendation pool (9) therein. Moreover, the memory unit (6) is configured such as to be able to record data on the processing unit (5) and to have an access to the prerecorded data.

Said reference chart (7) contains the reference data of the physical characteristics of the urine test strip (1) including the size of the urine test strip (1), and the location map of each reaction area (2) and etc. Said color scale (8) is used to generate a result by comparing the color changes occurred in the reaction areas (2) with the reference colors.

Said user data (10) contain the prerecorded user profiles, and also, they are configured such that the data and profiles of a new user can be added as well. The user profiles in the user data (10) includes the data on user's height, weight, age, possibility of any chronic disease, eating pattern, exercise rate, step count per day, blood glucose level, heartbeat rate, tension measurements, stress level, blood oxygen level, etc. Said recommendation pool (9) is a memory unit (6) part used in generating a recommendation by matching the data in the user profile contained in the user data (10) of the user subjected to the urine test with the urinalysis results.

In another embodiment of the invention, the urinalysis results of the user can be submitted to another mobile communication device (4), a server, etc. via the communication unit (11) of the mobile communication device (4).

In a further embodiment of the invention, said mobile communication device (4) can be electronic communication devices such as a mobile phone, a tablet, a laptop computer, etc.

In a different embodiment of the invention, said display (12) can be a touch screen configured such as to allow the user to input data.

In another embodiment of the invention, said user data (10) include the user profiles containing various data for different users. Thus, it can be used in giving recommendation to the user by obtaining data on age, gender, chronic diseases and the like of the user subjected to the urinalysis.

One of the most important advantages of the invention is that it can be used in any environment and without any extra costs. For example, it can be conveniently used in the disadvantaged regions having limited health care services, houses, and small-sized hospitals without laboratories. This is particularly important related to people with a chronic disease and consistently in need for the urinalysis. Furthermore, the urinalysis results of the urine analyzed by using the data provided in the memory unit (6) by unskilled persons in the art can be obtained as well.

Moreover, thanks to the invention, the urinalysis results recorded on the user profiles can be monitored retrospectively. Thus, the user is easily able to have an access to his/her previous urinalysis results.

1. A method of analyzing a urinalysis, characterized in that it comprises the steps of,
pouring a urine sample of a user subjected to a urinalysis over at least reaction areas (2) of a urine test strip (1) such that it is in contact with said reaction areas,
taking an image of the urine test strip (1) enabled to contact with the urine with an image capturing unit,
transmitting the image of the urine test strip (1) taken, to a processing unit (5),
recognizing the urine test strip (1) whose image is rendered by using the data in a reference chart (7) provided in a memory unit (6) by the processing unit (5) in the pattern of two columns and five rows,
determining ten reaction areas (2) on the strip,
determining the color changes occurred in the reaction area (2) with the image rendering technology without any reference by using more than one color space together,
obtaining a urinalysis result by matching the data on the color changes determined with the data contained within a color scale (8) of the memory unit (6) in the processing unit (5),
receiving the data of the user subjected to the analysis in the user profile from user data (10) contained within the memory unit (6) by means of the processing unit (5),
determining the recommendation in which the user profile data received and said urinalysis results are matched in a recommendation pool (9),
submitting the recommendation determined to the user.

2. A method according to the claim 1, characterized in that the urine test strip (1) used in the method enables using the image rendering technology without any reference by using more than one color space together.

3. A method according to the claim 1, characterized in that the urine test strip (1) used in the method comprises the reaction areas (2) formed of two columns and five rows and a black frame.

4. A method according to the claim 1, characterized in that the mobile communication device (4) used in the method comprises,
an image capturing unit (13) enabling an image of the strip (1) to be taken,
a memory unit (6) containing the predetermined user data (10) and the data on the reference chart (7), the color scale (8) and the recommendation pool (9),
a processing unit (5) generating a urinalysis result by comparing the colors of the reaction area (2) provided on the image of the urine test strip (1) received from the image capturing unit (13) with the data on the color scale (8) provided on the memory unit (6) as well as by detecting the matches,
a display (12) through which the results generated in the processing unit (5) are delivered to the user.
